SEQUENCE LISTING (36 sequences)

SEQ ID NO: 1 - DNA, 26 bp, artificial sequence
  Primer Pab154
accactcctttttctttttggctcat

SEQ ID NO: 2 - DNA, 26 bp, artificial sequence
  Primer Pab156
acctccaatcaaaatgtccagttcag

SEQ ID NO: 3 - DNA, 93 bp, artificial sequence
  Primer B438
gactgaattcggatccgtcgacgaagttcctattccgaagttcctattctctagaaagtataggaacttcctgggaccaataataatgactag

SEQ ID NO: 4 - DNA, 105 bp, artificial sequence
  Primer B439
gactaagcttggatcctctagagtcgacgcttagcgaagttcctatactatttgaagaataggaacttcggaataggaacttcgactgtaaaaagtacagtcggc

SEQ ID NO: 5 - DNA, 41 bp, artificial sequence
  Primer B452
gactgaattccatggtatcagtttgaaaattatgtattatg

SEQ ID NO: 6 - DNA, 43 bp, artificial sequence
  Primer B453
gactaagcttggatccatgggaagtctggtctcttaaagaaaa

SEQ ID NO: 7 - DNA, 758 bp, artificial sequence
  Sequence of the dsRED containing 750 bp NcoI fragment present in pSJ13012.
ccatgggaagtctggtctcttaaagaaaaagatgatgccttccttcagttcgctcatactgctcaacaattgtgtagtcttcgttgtgactagttatgtcaagttttgaatcaacatagtagtagccaggaagctgcacaggctttttcgccatgtaaatgctcttgaactcaacaagataatgccctccatctttcaactttaaggctttgtgaatttcacctttcaagacaccatcacgcggataaagcctctcagttgacggctcccaacccattgtctttttctgcattacaggaccatcacttggaaagttcacgccaatgaactttactttgtaaatgaagcaaccgtcttgcagacttgaatcttgcgttacagttacgacacctccatcttcaaagttcattacgcgctcccacttgaatccttcaggaaaacttaactttttgtaatcaggaatgtcagcaggatgctttacataaacttttgacccatactgaaactgcggacttaagatgtcccaagcaaacggcagcggtcctccttttgtcactttaagttttgcagtttgcgttccttcataaggccttccttcaccttcaccttcaatttcaaactcatgcccgtttacacttccttccattcgcactttgaagcgcatgaactctttgattacgtcttcagttgaagccattcggttccctcctcatttttatagagctccataatacataattttcaaactgataccatgg

SEQ ID NO: 8 - DNA, 48 bp, artificial sequence
  Primer B448
gactgaattccatggcaattgcgtataataaagaataattattaatct

SEQ ID NO: 9 - DNA, 51 bp, artificial sequence
  Primer B449
gactaagcttcaattggatccatggactaaattaaagtaataaagcgttct

SEQ ID NO: 10 - DNA, 43 bp, artificial sequence
  Primer B450
gactgaattccatggcaattgggccagtttgttgaagattaga

SEQ ID NO: 11 - DNA, 50 bp, artificial sequence
  Primer B451
gactaagcttcaattggatccatggccaacatgattaacaattattagag

SEQ ID NO: 12 - DNA, 95 bp, artificial sequence
  Primer B435
gactgaattcggatccgtcgacgaagttcctattccgaagttcctattctctagaaagtataggaacttcataaatgagtagaaagcgccatatc

SEQ ID NO: 13 - DNA, 106 bp, artificial sequence
  Primer B436
gactaagcttggatcctctagagtcgacgcttagcgaagttcctatactttctagagaataggaacttcggaataggaacttcctttagtgtcaattggaagtctg

SEQ ID NO: 14 - DNA, 1065 bp, artificial sequence
  Sequence of the FRT-F - PamyL_4199-dsRED - FRT-F containing SalI insert in pSJ13046.
gtcgacgcttagcgaagttcctatactttctagagaataggaacttcggaataggaacttcctttagtgtcaattggaagtctggtctcttaaagaaaaagatgatgccttccttcagttcgctcatactgctcaacaattgtgtagtcttcgttgtgactagttatgtcaagttttgaatcaacatagtagtagccaggaagctgcacaggctttttcgccatgtaaatgctcttgaactcaacaagataatgccctccatctttcaactttaaggctttgtgaatttcacctttcaagacaccatcacgcggataaagcctctcagttgacggctcccaacccattgtctttttctgcattacaggaccatcacttggaaagttcacgccaatgaactttactttgtaaatgaagcaaccgtcttgcagacttgaatcttgcgttacagttacgacacctccatcttcaaagttcattacgcgctcccacttgaatccttcaggaaaacttaactttttgtaatcaggaatgtcagcaggatgctttacataaacttttgacccatactgaaactgcggacttaagatgtcccaagcaaacggcagcggtcctccttttgtcactttaagttttgcagtttgcgttccttcataaggccttccttcaccttcaccttcaatttcaaactcatgcccgtttacacttccttccattcgcactttgaagcgcatgaactctttgattacgtcttcagttgaagccattcggttccctcctcatttttatagagctccataatacataattttcaaactgataaaatgatttttcataaatccattagacggtgcaaatatatgtttttaatgttcttcgtttttaggcatccctcctttcaatgtgatacatatgatattgtataaatattccgaatttttaacaagtaccattttccctatattttcttccaaaagaaaagcgccgatatggcgctttctactcatttatgaagttcctatactttctagagaataggaacttcggaataggaacttcgtcgac

SEQ ID NO: 15 - DNA, 106 bp, artificial sequence
  Primer B437
gactaagcttggatcctctagagtcgacgcttagcgaagttcctatactatttgaagaataggaacttcggaataggaacttcctttagtgtcaattggaagtctg

SEQ ID NO: 16 - DNA, 1386 bp, artificial sequence
  DNA segment containing the mRNA stabilizing element from the Bacillus thuringiensis cry3A gene, followed by a FRT-F site, a green-fluorescent protein (gfp) CDS with a ribosome binding site, and a FRT-F3 site (see figure 1).
gaattctaaagataatatctttgaattgtaacccccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggaagttcctattccgaagttcctattctctagaaagtataggaacttcaaaatgagagggagaggaaactcatgagtaaaggagaagaacttttcactggagttgtcccaattcttgttgaattagatggcgatgttaatgggcaaaaattctctgttagtggagagggtgaaggtgatgcaacatacggaaaacttacccttaaatttatttgcactactgggaagctacctgttccatggccaacgcttgtcactactctcacttatggtgttcaatgcttttctagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaattataactcacataatgtatacatcatggcagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacgcaatctgccctttccaaagatcccaacgaaaagagagatcacatgatccttcttgagtttgtaacagctgctgggattacacatggcatggatgaactatacaaataagaagttcctattccgaagttcctattcttcaaatagtataggaacttcaagctt

SEQ ID NO: 17 - DNA, 20 bp, artificial sequence
  Primer 411451
gtccgaatcccgctacaacg

SEQ ID NO: 18 - DNA, 20 bp, artificial sequence
  Primer 411452
caatgacgtgacgtgttgcc

SEQ ID NO: 19 - DNA, 25 bp, artificial sequence
  Primer B335
ttctattggaatgattaagattcca

SEQ ID NO: 20 - DNA, 23 bp, artificial sequence
  Primer B471
gcaccgtctaatggatttatgaa

SEQ ID NO: 21 - DNA, 22 bp, artificial sequence
  Primer B533
cttcattgcggaatgaacaagc

SEQ ID NO: 22 - DNA, 22 bp, artificial sequence
  Primer B539
ttgcccgaatacaacgacaggc

SEQ ID NO: 23 - DNA, 1410 bp, artificial sequence
  Nucleotide sequence of P33JVS_r4_fl
gactggatccgaattccaattgaattatggccacattgaaaggggaggagaatcatgccccaattcgatatcctgtgcaagacacctccgaaggtgctggtgaggcagttcgtggaacgtttcgaaagaccgagtggggaaaagatcgcgctgtgtgcagcggaactgacatatctgtgctggatgatcactcataacggtacagcgatcaaaagagcgactttcatgagctataacacaatcatctcaaactcactgtcatttgatatcgtgaacaagtccctgcagtttaagtacaagacacagaaggcgacgatcctggaagcgtcgctgaagaagctgattcctgcgtgggagtttaccatcatcccgtactatggacaaaagcatcaaagcgacatcacagatatcgtgtcttccctgcaactgcaatttgaatcgagcgaagaggcggataagggaaacagtcactctaagaagatgctgaaggcgctgctgagcgaaggcgaaagcatctgggagatcacagaaaagatcctgaacagctttgagtatacgagtagatttaccaagacgaagacgctgtatcaattcctgtttctggcgacattcatcaactgtggaaggttctctgatatcaagaacgtggaccccaagagctttaagctggtgcagaacaagtacctgggggtgatcatccaatgtctggtgacagagacaaagaccagtgtgtcacgccacatctacttcttttcagcgagaggaaggatcgatccgctggtgtacctggatgaatttctgcgaaacagcgaacccgtgctgaagagagtgaaccgcacaggcaactcaagttcgaacaagcaagagtatcaactgctgaaggataacctggtgcgttcgtataacaaggcgctgaagaagaatgcaccgtactctatctttgcgatcaagaacggtccgaagagtcatatcggtaggcatctgatgacgagctttctgagcatgaagggcctgacggaactgacgaacgtggtgggaaactggtccgataagcgagcgtctgcggtggcacggacaacgtacacacaccagatcacagcgatccctgaccactactttgcgctggtgtccaggtactatgcgtatgatccaatcagcaaggagatgatcgcgctgaaggacgagacgaaccccatcgaagaatggcagcatatcgaacaactgaaggggtcagcggagggctcgatccgctatcctgcgtggaacggaatcatctcccaagaagtgctggactatctgagcagctacatcaacagacgcatctagaagagcagagaggacggatttcctgaaggaaatccgtttttttattttgcacgcgtgctagcaagcttgaattcggatccgact

SEQ ID NO: 24 - DNA, 1410 bp, artificial sequence
  Nucleotide sequence of D438GY_fl
gactggatccgaattccaattgaattatggccacattgaaaggggaggagaatcatgccgcaatttgatatcctgtgcaagacacctccgaaggtgctggtgcggcaatttgtggaaaggtttgaaagaccgagcggtgaaaagatcgcgctgtgtgcagcggaactgacttatctgtgctggatgatcacacataacggaactgcgatcaaaagagcgacattcatgtcatacaacacaatcatctctaacagcctgtcgtttgatatcgtgaacaagtcgctgcagtttaagtacaagacgcaaaaggcgacaatcctggaagcgtccctgaagaagctgatcccagcgtgggagtttacgatcatcccgtattacggccagaagcaccagagcgacatcacagatatcgtgtcttcactgcaactgcaattcgaaagttcggaagaagcggataagggaaactctcattcgaagaagatgctgaaggcgctgctgagcgaaggcgaatcgatctgggagatcacggaaaagatcctgaactctttcgagtacactagccggttcactaagactaagacactgtatcaatttctgtttctggcgacctttatcaactgtggaagattctcagacatcaagaacgtggacccgaagtcgtttaagctggtgcagaacaagtatctgggagtgatcatccaatgcctggtgacagaaactaagacgtcggtgtccaggcatatctactttttctccgcgagaggaagaatcgatccactggtgtatctggatgaatttctgcggaactccgaaccggtgctgaagcgtgtgaaccgcacaggaaacagttcctcaaacaagcaggaatatcagctgctgaaggataacctggtgagatcatacaacaaggcgctgaagaagaatgcaccgtacagcatcttcgcgatcaagaacggacctaagagccatatcggacgccatctgatgacttcctttctgtcaatgaagggtctgactgaactgacaaacgtggtggggaactggtccgacaaaagagcgtcagcggtggcacggaccacttatacccaccagatcactgcgatcccggatcactactttgcgctggtgagccgctactatgcgtatgatcctatcagcaaggaaatgatcgcgctgaaggacgaaacaaacccgatcgaggaatggcagcatatcgaacaactgaagggctcagcggaaggatcgatcagatatcctgcgtggaacggaatcatctcacaggaagtgctggattacctgtcaagctatatcaacagacgcatctagaagagcagagaggacggatttcctgaaggaaatccgtttttttattttgcacgcgtgctagcaagcttgaattcggatccgact

SEQ ID NO: 25 - DNA, 22 bp, artificial sequence
  Primer B440
gactggatccgaattccaattg

SEQ ID NO: 26 - DNA, 23 bp, artificial sequence
  Primer B441
agtcggatccgaattcaagcttg

SEQ ID NO: 27 - DNA, 24 bp, artificial sequence
  Primer B317
gttttatgtttcggtataaaacac

SEQ ID NO: 28 - DNA, 23 bp, artificial sequence
  Primer B321
tattccatggacttcatttactg

SEQ ID NO: 29 - DNA, 3598 bp, artificial sequence
  Exemplary DNA sequence of a FRT-F and FRT-F3 flanked RFP expression construct integrated at the bglC locus
atggagcatatatcctggcttacatttacgatgctgattctcgccagttaccggttaactcatttgattgtttttgataagatcacggagtttatccggaaaccgttcatgaagaagaagcagacgattgatgaacaagggcatgtagaaacgaaaaaagtgccgaaatcaaacttcggctatttgctgaattgctattggtgcgcagggatatggtgcgcgttgatcattgctgtcggatatctgattgccccaaaagcgatattcccgttgattttgattttgtcggtcgccggggggcaggcgattcttgaaacgtttgtcggtgtcgccacaaaacttgtcggctttttctccgatttaaagaagtaaaccattccaagcggatggttttatttttttgtcaataaagtgatacaaacagcagagagaacgtgtcagttttatgaacttttcacagcgatttttcccggatgcggcattttaggcagagaggaagcatctcattgtaaagatttcagtttttaaaatttagaattgagagaaaaaggatgtgcaaagtccccggagctcggatccactagtaacggccgccagtgtgctggaattcgcccttgcggccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgtggccagaagttcctattccgaagttcctattctctagaaagtataggaacttcttataaaaatgaggagggaaccgaatggcttcaactgaagacgtaatcaaagagttcatgcgcttcaaagtgcgaatggaaggaagtgtaaacgggcatgagtttgaaattgaaggtgaaggtgaaggaaggccttatgaaggaacgcaaactgcaaaacttaaagtgacaaaaggaggaccgctgccgtttgcttgggacatcttaagtccgcagtttcagtatgggtcaaaagtttatgtaaagcatcctgctgacattcctgattacaaaaagttaagttttcctgaaggattcaagtgggagcgcgtaatgaactttgaagatggaggtgtcgtaactgtaacgcaagattcaagtctgcaagacggttgcttcatttacaaagtaaagttcattggcgtgaactttccaagtgatggtcctgtaatgcagaaaaagacaatgggttgggagccgtcaactgagaggctttatccgcgtgatggtgtcttgaaaggtgaaattcacaaagccttaaagttgaaagatggagggcattatcttgttgagttcaagagcatttacatggcgaaaaagcctgtgcagcttcctggctactactatgttgattcaaaacttgacataactagtcacaacgaagactacacaattgttgagcagtatgagcgaactgaaggaaggcatcatctttttctttaagaagttcctattccgaagttcctattcttcaaatagtataggaacttcacgcgtagggcccgcggctagcggccgcgtcgactagaagagcagagaggacggatttcctgaaggaaatccgtttttttattttgcccgtcttataaatttcgttgtccaactcgcttaattgcgagtttttatttcgtttatttcaatcaaggtaaatggctagcggccgcgtcgactagaagagcagagaggacggatttcctgaaggaaatccgtttttttattttgcccgtcttataaatttcgttgccatgggatccgcggccgcgctgcagccaacacgatagcagtacaatacagagcgggggacaacaatgtaaacggcaaccaaatccgccctcagctcaacattaaaaacaacagcaaaaaaaccgtctctttaaatcgaatcaccgtccgctactggtataaaacgaatcgcaaaggaaaaaattttgactgcgactatgcccaaatcggctgcagcaaaatcacgcacaaattcgtccaattaaaaaaagcggtaaacggagcagacacgtatcttgaagtagggtttaaaaatggtacattggcgccgggtgcaagtacaggtgaaatccagatccgtcttcacaatgacggctggagcaattatgcccaaagcggcgactattcatttttaaattcaaacacgtttaaaaatacgaaaaaaatcacgttgtatgagaacggaaagctgatttggggcactgaacctaaataacggcactttgacggacaccggatttggtgtccgttttcgtatatattataatggaaggaatgaggaatatttttgtaaacatgaaaggagatggatgtatgaatgaaacattgcagcaatacatgatgcttgtcaaggaacactatgacacgatcaatggaccggattacacaggcaaagaggaagacattgaaaagagaaaagagcaaatcgagctttacgccaaaacgctccagcaaggcttttcaacagatgacgactatgatgaattcgcagatgccgtgattaaatgcgcatacggagatctgacgatggaagaattggaaacggtttatcgggaattaacgtctccataa

SEQ ID NO: 30 - DNA, 37 bp, artificial sequence
  Primer B651
gactagatctgaattctgctgtccagactgtccgctg

SEQ ID NO: 31 - DNA, 47 bp, artificial sequence
  Primer B653
gactagatctggccacattttcttatacaaattatattatacatatc

SEQ ID NO: 32 - DNA, 20 bp, artificial sequence
  Primer B622
agtatcatattgacggcttc

SEQ ID NO: 33 - DNA, 54 bp, artificial sequence
  Primer B664
aatttgtataagaaaatgtggccagatctagtcccacattgaaaggggaggaga

SEQ ID NO: 34 - DNA, 5998 bp, artificial sequence
  pSJ13654
aattcagatctgaattctgctgtccagactgtccgctgtgtaaaaaaaaggaataaaggggggttgacattattttactgatatgtataatataatttgtataagaaaatgtggccacattgaaaggggaggagaatcatgccgcaatttgatatcctgtgcaagacacctccgaaggtgctggtgcggcaatttgtggaaaggtttgaaagaccgagcggtgaaaagatcgcgctgtgtgcagcggaactgacttatctgtgctggatgatcacacataacggaactgcgatcaaaagagcgacattcatgtcatacaacacaatcatctctaacagcctgtcgtttgatatcgtgaacaagtcgctgcagtttaagtacaagacgcaaaaggcgacaatcctggaagcgtccctgaagaagctgatcccagcgtgggagtttacgatcatcccgtattacggccagaagcaccagagcgacatcacagatatcgtgtcttcactgcaactgcaattcgaaagttcggaagaagcggataagggaaactctcattcgaagaagatgctgaaggcgctgctgagcgaaggcgaatcgatctgggagatcacggaaaagatcctgaactctttcgagtacactagccggttcactaagactaagacactgtatcaatttctgtttctggcgacctttatcaactgtggaagattctcagacatcaagaacgtggacccgaagtcgtttaagctggtgcagaacaagtatctgggagtgatcatccaatgcctggtgacagaaactaagacgtcggtgtccaggcatatctactttttctccgcgagaggaagaatcgatccactggtgtatctggatgaatttctgcggaactccgaaccggtgctgaagcgtgtgaaccgcacaggaaacagttcctcaaacaagcaggaatatcagctgctgaaggataacctggtgagatcatacaacaaggcgctgaagaagaatgcaccgtacagcatcttcgcgatcaagaacggacctaagagccatatcggacgccatctgatgacttcctttctgtcaatgaagggtctgactgaactgacaaacgtggtggggaactggtccgacaaaagagcgtcagcggtggcacggaccacttatacccaccagatcactgcgatcccggatcactactttgcgctggtgagccgctactatgcgtatgatcctatcagcaaggaaatgatcgcgctgaaggacgaaacaaacccgatcgaggaatggcagcatatcgaacaactgaagggctcagcggaaggatcgatcagatatcctgcgtggaacggaatcatctcacaggaagtgctggattacctgtcaagctatatcaacagacgcatctagagagaggacggatttcctgaaggaaatccgtttttttattttgcacgcgtgctagcggccgcgtcgacgaagttcctattccgaagttcctattctctagaaagtataggaacttcagaagagctgcatcaaaaatcggagccgaagttttgaaaaagggcgggaatgccattgatgcagctattgcgagctcttccaggaagttcctattccgaagttcctattcttcaaatagtataggaacttcaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 35 - DNA, 35 bp, artificial sequence
  Primer B692
gactgctcttccttcattgaaaggggaggagaatc

SEQ ID NO: 36 - DNA, 35 bp, artificial sequence
  Primer B693
gactgctcttctcctctatctttgaacataaattg

(19) United States
(12) Patent Application Publication
(10) Pub. No.: US 2019/0276855 A1
(43) Pub. Date: Sep. 12, 2019

(54) FLP-MEDIATED GENOMIC INTEGRATION IN BACILLUS LICHENIFORMIS

(71) Applicant: Novozymes A/S, Bagsvaerd (DK)

(72) Inventor: Steen Troels Joergensen, Alleroed (DK)

(73) Assignee: Novozymes A/S, Bagsvaerd (DK)

(21) Appl. No.: 16/345,219

(22) PCT Filed: Oct. 23, 2017
(86) PCT No.: PCT/EP2017/076992

§ 371 Date: Apr. 25, 2019

(30) Foreign Application Priority Data
Oct. 25, 2016 (EP) 16195510.9

(51) Int. Cl. C12N 15/90; C12N 15/75

(52) U.S. Cl. CPC C12N 15/902; C12N 15/75

(57) ABSTRACT

The present invention relates to methods for the site-specific integration of at least one polynucleotide of interest into the chromosome of a Bacillus licheniformis host cell using the FLP/FRT system derived from Saccharomoces cerevisiae or a homologue or variant thereof.

### REFERENCE TO A SEQUENCE LISTING

This application contains a Sequence Listing in computer readable form, which is incorporated herein by reference.

### BACKGROUND OF THE INVENTION

A large number of naturally-occurring organisms have been found to produce useful polypeptide products, e.g., enzymes, the large scale production of which is desirable for research and commercial purposes. Once such a polypeptide product has been identified, efforts are often made to develop manufacturing methods having an improved productivity. One widely used method, which is based on recombinant DNA techniques, is to clone a gene encoding the product and insert the gene into a suitable expression system in order to express the product in a suitable host cell, either integrated in the chromosome or as an extrachromosomal entity, under conditions conducive for the expression of the product.

Irrespective of which production method is used, it is normally desirable to increase the production level of a given polypeptide or protein. Thus, efforts are being made to increase the production, e.g. by inserting the gene encoding the product under the control of a strong expression signal, increasing the stability of the trancribed mRNA or by increasing the number of copies of the gene in the production organism in question. This latter approach may be accomplished by inserting the gene into a multicopy plasmid which generally, however, tends to be unstable in the host cell in question, or by integrating multiple copies of the gene into the chromosome of the production organism, an approach which generally is considered more attractive because the stability of the construct tends to be higher.

Construction of host cells has been described, wherein a highly expressed chromosomal gene is replaced with a recognition sequence of a site-specific recombinase to allow subsequent insertion of a single product-encoding polynucleotide into that site by the use of a recombinase recognizing said sequence (EP 1 405 908 A1; ProBioGen AG).

It has been disclosed to insert DNA at a known location in the genome (O'Gorman et al., 1991 Science, 251:1351-55; Baubonis and Sauer, 1993 Nucl., Acids Res., 21:2025-29; Albert et al., 1995 Plant J., 7:649-59). These methods make use of site-specific recombination systems that are freely reversible. These reversible systems include the following: the Cre-lox system from bacteriophage P1 (Baubonis and Sauer, 1993, supra; Albert et al., 1995 Plant J., 7549-59), the FLP-FRT system of Saccharomyces cerevisiae (O'Gorrnan et al., 1991, supra), the R-RS system of Zygosaccharonzyces rouxii (Onouchi et al., 1995 Mol. Gen. Genet. 247: 653-660), a modified Gin-gix system from bacteriophage Mu (Maeser and Kahmann, 1991 Mol. Gen. Genet., 230: 170-76), the beta-recombinase-six system from a Bacillus subtilis plasmid (Diaz et al., 1999 J. Biol. Chem. 274: 6634-6640), and the delta-gamma-res system from the bacterial transposon Tn1000 (Schwikardi and Dorge, 2000 E B S let. 471: 147-150). Cre, FLP, R, Gin, beta-recombinase and gamma-delta are the recombinases, and lox, FRT, RS, gix, six and res the respective recombination sites (reviewed by Sadowslu, 1993 FASEB J., 7:750-67; Ow and Medberry, 1995 Crit. Rev. Plant Sci. 14: 239-261).

Multiplex Cre/lox recombination permits selective site-specific DNA targeting to both a natural and an engineered site in the yeast genome (Sauer, B. Nucleic Acids Research. 1996, Vol. 24(23): 4608-4613). It has been shown that infection of host cells having a natural attachment site, attB as well as an ectopically introduced attB site, with a derivative of the Streptomyces phage ΦC31, resulted in the integration of the phage into both attB sites (Smith et al. 2004. Switching the polarity of a bacteriophage integration system. Mol Microbiol 51(6):1719-1728). Multiple copies of a gene can be introduced into a cell comprising multiple attachment sites recognized by the Mx9 integrase using the Mx9 phage transformation system, (WO 2004/018635 A2). The temperal Lactococcal bacteriophage TP901-1 integrase and recognition sequences are well-characterized (Breüner et al. (1990) Novel Organization of Genes Involved in Prophage Excision Identified in the Temperate Lactococcal Bacteriophage TP901-1. J Bacteriol 181(23): 7291-7297; Breüner et al. 2001. Resolvase-like recombination performed by the TP901-1 integrase. Microbiology 147: 2051-2063).

The site-specific recombination systems above have in common the property that a single polypeptide recombinase catalyzes the recombination between two sites of identical or nearly identical sequences. Each recombination site consists of a short asymmetric spacer sequence where strand exchange tales place, flanked by an inverted repeat where recombinases bind. The asymmetry of the spacer sequence gives an orientation to the recombination site, and dictates the outcome of a recombination reaction. Recombination between directly or indirectly oriented sites in cis excises or inverts the intervening DNA, respectively. Recombination between sites in trans causes a reciprocal translocation of two linear DNA molecules, or co-integration if at least one of the two molecules is circular. Since the product-sites generated by recombination are themselves substrates for subsequent recombination, the reaction is freely reversible. In practice, however, excision is essentially irreversible because the probability of an intramolecular interaction, where the two recombination-sites are closely linked, is much higher than an intermolecular interaction between unlinked sites. The corollary is that the DNA molecule inserted into a genomic recombination site will readily excise out.

The simultaneous genomic integration of multiple copies of a promoterless open reading frame or operon by the site-specific and transiently expressed temperal Lactococcal bacteriophage TP901-1 integrase has previously been shown in a Bacillus host (WO 2006/042548).

Still, Bacillus licheniformis is one of the preferred industrial production hosts for the manufacture of, e.g., enzymes, and efficient site-specific recombination systems suitable for use in B. licheniformis remain in high demand.

### FIELD OF THE INVENTION

The present invention relates to methods for the site-specific integration of at least one polynucleotide of interest into the chromosome of a Bacillus licheniformis host cell using the FLP/FRT system derived from Saccharomoces cerevisiae or a homologue or variant thereof.

### SUMMARY OF THE INVENTION

The present invention relates to methods for the site-specific integration of at least one polynucleotide of interest into the chromosome of a Bacillus licheniformis host cell, said method comprising the steps of:

(a) providing a B. licheniformis host cell comprising in its chromosome at least one integration site, each integration site comprising a pair of recognition sequences of the site-specific Flippase recombinase, FLP, from Saccharomyces cerevisiae, or a homologoue thereof;
(b) introducing into said cell a nucleic acid construct also comprising the pair of recognition sequences of the site-specific recombinase, said pair flanking the polynucleotide of interest;
(c) expressing the site-specific FLP recombinase or homologue thereof in the cell, whereby the at least one chromosomal recognition sequence pair is recombined with the corresponding recognition sequence pair of the nucleic acid construct by the FLP recombinase to produce a B. licheniformis host cell comprising at least one polynucleotide of interest site-specifically integrated into the chromosome of the cell.

### BRIEF DESCRIPTION OF THE FIGURE

FIG. 1 shows the DNA segment of Example 11 which contains the mRNA stabilizing element from the Bacillus thuringiensis cry3A gene, followed by a FRT-F site, a green-fluorescent protein (gfp) CDS with a ribosome binding site, and a FRT-F3 site; the nucleotide sequence is provided in SEQ ID NO:16.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

### DETAILED DESCRIPTION OF THE INVENTION

As already mentioned in the above, the present invention relates to methods for the site-specific integration of at least one polynucleotide of interest into the chromosome of a Bacillus licheniformis host cell, said method comprising the steps of:

(a) providing a B. licheniformis host cell comprising in its chromosome at least one integration site, each integration site comprising a pair of recognition sequences of the site-specific Flippase recombinase, FLP, from Saccharomyces cerevisiae, or a homologoue or variant thereof;
(b) introducing into said cell a nucleic acid construct also comprising the pair of recognition sequences of the site-specific recombinase, said pair flanking the polynucleotide of interest;
(c) expressing the site-specific FLP recombinase or homologue thereof in the cell, whereby the at least one chromosomal recognition sequence pair is recombined with the corresponding recognition sequence pair of the nucleic acid construct by the FLP recombinase to produce a B. licheniformis host cell comprising at least one polynucleotide of interest site-specifically integrated into the chromosome of the cell.

The site-specific recombinase and its pair of recognition sequences are from the Saccharomyces cerevisiae FLP-FRT system. In a particular embodiment, the FLP recombinase is the FLP recombinase variant as described in Buchholz, Frank, Improved properties of FLP recombinase evolved by cycling mutagenesis, Nature Biotechnology Volume: 16 Issue: 7 (1998-07-01) p. 657-662. In another particular embodiment, the FLP recombinase is a thermostable recombinase variant designated “FLPe” having amino acid alterations P2S, L33S, Y108N, S294P; the nucleic acid sequence and corresponding amino acid sequence for FLPe is shown in SEQ ID NO:106 and SEQ ID NO:107 of WO 2012/160093, respectively.

In a preferred embodiment of the first aspect, the polynucleotide of interest comprises an operon or an open reading frame encoding at least one polypeptide of interest.

Preferably, the polypeptide of interest comprises an enzyme, preferably a hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

In another preferred embodiment, a selection or screening marker is located in between the pair of recognition sequences in the at least one integration site; preferably the marker is a gene encoding a fluorescent polypeptide to enable the selection of a non-fluorescing cell, wherein the polynucleotide of interest has been integrated in every integration site.

Preferably, the nucleic acid construct further comprises an incoming selection marker and a polynucleotide encoding the FLP recombinase, or a homologue thereof.

Alternatively, it is preferably that a second nucleic acid construct is introduced into said cell in step (b) which is either non-replicating or temperature-sensitively replicating, and which comprises a polynucleotide encoding the FLP recombinase, or a homologue thereof, and a selection marker which enables positive or negative selection or is bi-directional, and which is maintained in said cell transiently by selective pressure or growth at the permissive temperature, respectively, so that the recombinase can be transiently expressed in step (c).

As a third preferred alternative embodiment, the cell in step (a) comprises in its chromosome at least one copy of a polynucleotide encoding the recombinase operably linked with a tightly regulated promoter, which can be turned on and off by changing the growth conditions, so as to enable the transient expression of the site-specific recombinase in step (c).

In a preferred embodiment, the pair of recognition sequences consists of two different recognition sequences, preferably the wildtype FRT sequence in combination with a derivative thereof, more preferably the wildtype FRT sequence in combination with a recognition sequence variant selected from the group consisting of FRT-F, FRT-F3, FRT-F10, FRT-F13, FRT-F14, FRT-F15, FRT-Fa and FRT-F3a. The FRT-F and FRT-F3 sequences are disclosed in WO 2012/160093. See also Turan et al., 2010, J. Mol. Biol. 402: 52-69, which is incorporated herein in its entirety.

It is clearly anticipated that the methods of the present invention will allow the simultaneous site-specific integration at several chromosomal integration sites of the B. licheniformis host cell to produce a multi-copy host cell.

Accordingly, in a preferred embodiment, the B. licheniformis host cell of step (a) comprises in its chromosome two or more integration sites and a B. licheniformis host cell comprising two or more polynucleotides of interest site-specifically integrated into the chromosome of the cell is produced; preferably the B. licheniformis host cell of step (a) comprises in its chromosome three or more integration sites; more preferably four or more, five or more, or even six or more integration sites, and wherein a B. licheniformis host cell comprising three, four, five, six or more polynucleotides of interest site-specifically integrated into the chromosome of the cell is produced.

### Nucleic Acid Constructs

The present invention also relates to nucleic acid constructs comprising a polynucleotide of interest operably linked to one or more control sequences that direct the expression of the encoded polypeptide of interest in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

### Expression Vectors

The present invention also relates to recombinant expression vectors comprising a polynucleotide of interest encoding a polypeptide of interest, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell.

Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant Bacillus licheniformis host cells, comprising one or more polynucleotide of interest operably linked to one or more control sequences that direct the production of a polypeptide of interest site-specifically integrated in its chromosome according to the methods of the present invention. A construct or vector comprising a polynucleotide is introduced into a host cell so that the construct or vector is maintained as a chromosomal integrant as described earlier. The term “host cell” encompasses any progeny of a parent cell that is not identical to the parent cell due to mutations that occur during replication. The choice of a host cell will to a large extent depend upon the gene encoding the polypeptide and its source.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

### Methods of Production

The present invention also relates to methods of producing a polypeptide of the present invention, comprising (a) cultivating a resulting recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Examples

### Materials and Methods

Chemicals used as buffers and substrates were commercial products of at least reagent grade.

PCR amplifications were performed using standard textbook procedures, employing a commercial thermocycler and either Ready-To-Go PCR beads, Phusion polymerase, or RED-TAQ polymerase from commercial suppliers.

LB agar: See EP 0 506 780.

LBPSG agar plates contains LB agar supplemented with phosphate (0.01 M K3PO4), glucose (0.4%), and starch (0.5%); See EP 0 805 867 B1.

TY (liquid broth medium; See WO 94/14968, p 16.

Oligonucleotide primers were obtained from DNA technology, Aarhus, Denmark. DNA manipulations (plasmid and genomic DNA preparation, restriction digestion, purification, ligation, DNA sequencing) were performed using standard textbook procedures with commercially available kits and reagents.

Ligation mixtures were in some cases amplified in an isothermal rolling circle amplification reaction, using the TempliPhi kit from GE Healthcare.

DNA was introduced into E. coli using chemically competent cells or electroporation, following textbook or manufacturers procedures.

DNA was introduced into B. subtilis rendered naturally competent, either using a two step procedure (Yasbin et al., 1975, J. Bacteriol. 121: 296-304.), or a one step procedure, in which cell material from an agar plate was resuspended in Spizisen 1 medium (WO 2014/052630), 12 ml shaken at 200 rpm for appr. 4 hours at 37° C., DNA added to 400 microliter aliquots, and these further shaken 150 rpm for 1 hour at the desired temperature before plating on selective agar plates.

DNA was introduced into B. licheniformis by conjugation from B. subtilis, essentially as prevously described (EP2029732 B1), using a modified B. subtilis donor strain PP3724, containing pLS20, wherein the methylase gene M.bli1904II (US20130177942) is expressed from a triple promoter at the amyE locus, the pBC16-derived orf beta and the B. subtilis comS gene (and a kanamycin resistance gene) are expressed from a triple promoter at the air locus (making the strain D-alanine requiring), and the B. subtilis comS gene (and a cat gene) are expressed from a triple promoter at the pet locus.

Bacillus subtilis JA1343: JA1343 is a sporulation negative derivative of PL1801 (WO 2005042750). Part of the gene spollAC has been deleted to obtain the sporulation negative phenotype.

Escherichia coli TG1: TG1 is a commonly used cloning strain and was obtained from a commercial supplier; it has the following genotype: F′[traD36 laclq A(lacZ) M15 proA+B+] glnV (supE) thi-1 A(mcrB-hsdSM)5 (rK-mK-McrB-) thi A(lac-proAB).

Plasmid pPP4758 was used as a source for the dsRED gene. It is an E. coli/B. subtilis shuttle vector composed of pUC19 and pE194 vector fragments, and carries an amyL-promoter derivative-dsRED construct, from which segments were cloned by PCR. The DNA sequences of these fragments are given in the individual examples, where relevant.

### Example 1. Construction of a Restriction-Deficient B. licheniformis Host Strain

Bacillus licheniformis strain SJ8071 (WO2007/138049; Example 1) was used as host for creation of a restriction-deficient strain.

Briefly, deletion plasmid pMDT139 (U.S. Pat. No. 7,820,408) was introduced into SJ8071 by conjugation from SJ8105 (conjugative donor strain PP289-5 containing pMDT139), essentially as prevously described (WO2007/138049), TetS transconjugants were isolated, they were plated on LBPGS plates with erythromycin (5 microgram/ml) at 50° C., colonies formed at 50° C. were spread on LBPGS plates without antibiotics and propagated at 34° C., and checked for ErmS by further reisolation and replica plating. Erythromycin-sensitive colonies were checked for presence of the Bli1904II deletion by PCR using standard textbook procedures, with primers pab154 and pab156 and an annealing temperature of 69° C. Among 60 colonies tested, 4 were found to harbour the deletion. Two deletion strains were kept as SJ12850 and SJ12851.

Primer Pab154:
(SEQ ID NO: 1)
5′-ACCACTCCTTTTTCTTTTTGGCTCAT
Primer Pab156:
(SEQ ID NO: 2)
5′-ACCTCCAATCAAAATGTCCAGTTCAG

### Example 2. Construction of Vector pSJ12964 Carrying FRT-F-Cat-FRT-F3

To have the cat gene flanked by FRT-F and FRT-F3 sites, oligonucleotides incorporating these sites were designed for amplification of the cat gene.

As source of the cat gene, originally derived from pC194, strain PP4573 was used. This strain contains a chromosomally integrated cat gene cloned from pDN1050 (Diderichsen et al., 1993, Plasmid 30(3): 312-315). pDN1050 has the DNA sequence given in EMBL:Z22671, and the region, that after amplification as described below becomes inserted between the FRT-F and FRT-F3 sites extends from pos. 444 to pos. 1334 in Z22671.

Oligonucleotide #B438 is a forward primer for the cat gene segment, incorporating a FRT-F site and restriction sites EcoRI, BamHI, and SalI for cloning, whereas oligonucleotide #B439 is a reverse primer for the cat gene segment, incorporating a FRT-F3 site and restriction sites HindIII, BamHI, XbaI, SalI, and EspI for cloning.

Primer #B438:
(SEQ ID NO: 3)
5′-GACTGAATTCGGATCCGTCGACGAAGTTCCTATTCCGAAGTTCCTAT
TCTCTAGAAAGTATAGGAACTTCCTGGGACCAATAATAATGACTAG
Primer #B439:
(SEQ ID NO: 4)
5′-GACTAAGCTTGGATCCTCTAGAGTCGACGCTTAGCGAAGTTCCTATA
CTATTTGAAGAATAGGAACTTCGGAATAGGAACTTCGACTGTAAAAAGTA
CAGTCGGC

Plasmid pSJ8017 (EP2029732B1) is a temperature-sensitive replicon carrying 3′ and 5′ segments from the B. licheniformis cat locus (catL), and has been used to introduce a deletion of the licheniformis cat gene. It may also be used to insert heterologous DNA at the chromosomal catL locus, by double homologous recombination.

To insert a cassette consisting of FRT-F-cat-FRT-F3 into this plasmid, between the licheniformis 3′ and 5′ catL segments, the cat gene from strain PP4573 was PCR amplified using primers B438+B439, and the resulting appr. 1 kb fragment digested with SalI. This SalI fragment was ligated to the 5.3 kb SalI digested pSJ8017, the ligation mixture introduced into B. subtilis JA1343 competent cells, and a resulting strain kept as SJ12964 (JA1343/pSJ12964). The correctness of the PCR amplified segment was verified by DNA sequencing.

A strain suitable for transfer of pSJ12964 by conjugation was constructed by introduction of pSJ12964 into B. subtilis PP3724 naturally competent cells, selecting for erythromycin resistance (2 microgram/ml). 4 colonies were pooled and frozen as SJ12989=P P3724/pSJ12964.

### Example 3. Construction of Vectors pSJ13011 to pSJ13015 Carrying FRT-F-Cat::dsRED-FRT-F3 Between catL Segments

pPP4758 was used as source for the dsRED gene. In one approach to have the dsRED gene flanked by FRT-F and FRT-F3 sites, the dsRED gene was inserted into (and thus inactivating) the FRT-flanked cat gene in pSJ12964. The dsRED gene was PCR amplified from plasmid pPP4758 using primers #B452+#B453, which incorporated a NcoI site at either end of the amplified fragment.

Primer B452:
(SEQ ID NO: 5)
5′-GACTGAATTCCATGGTATCAGTTTGAAAATTATGTATTATG
Primer B453:
(SEQ ID NO: 6)
5′-GACTAAGCTTGGATCCATGGGAAGTCTGGTCTCTTAAAGAAAA

The approx. 750 bp amplified fragment was digested with NcoI and ligated to the NcoI digested pSJ12964 which has a unique NcoI site within the cat gene. The ligation mixture was treated (amplified) using a TempliPhi polymerase kit according to the manufacturers instruction and this amplified ligation mixture was used for transformation of B. subtilis JA1343 competent cells. Red coloured colonies were isolated and their plasmids analysed by restriction digestion. 5 clones were kept: SJ13011 (JA1343/pSJ13011), SJ13012 (JA1343/pSJ13012), SJ13013 (JA1343/pSJ13013), SJ13014 (JA1343/pSJ13014), and SJ13015 (JA1343/pSJ13015).

Plasmid pSJ13013 seems to have a double insert in one of the possible orientations, pSJ13012 and pSJ13014 seem to have a single insert in the other orientation, and pSJ13011 and pSJ13015 seem to have a double insert in this other orientation.

The DNA sequence of the dsRED containing 750 bp NcoI fragment, as present in pSJ13012, is shown in SEQ ID NO:7.

Strains suitable for transfer of pSJ13011 to pSJ13015 by conjugation were constructed by introduction of the plasmids into B. subtilis PP3724 competent cells, selecting erythromycin resistance (2 microgram/ml). From each transformation, 4 colonies were pooled and frozen as follows:
SJ13037=PP3724/pSJ13011.
SJ13038=PP3724/pSJ13012.
SJ13039=PP3724/pSJ13013.
SJ13040=PP3724/pSJ13014.
SJ13041=PP3724/pSJ13015.

### Example 4. Construction of Vectors pSJ13016 and pSJ13017, Carrying FRT-F-Cat::Spc-FRT-F3 Between catL Segments

In one approach to have a spc gene (conferring spectinomycin resistance) flanked by FRT-F and FRT-F3 sites, the spc gene was inserted into (and thus inactivating) the FRT-flanked cat in pSJ12964, using the unique MfeI and NcoI sites within the cat gene. The spc gene was PCR amplified from plasmid pSJ3358 (U.S. Pat. No. 5,882,888) using primers #6448+#6449, which incorporated an upstream MfeI and a downstream NcoI site in the amplified fragment.

Primer B448:
(SEQ ID NO: 8)
5′-GACTGAATTCCATGGCAATTGCGTATAATAAAGAATAATTATTAAT
CT
Primer B449:
(SEQ ID NO: 9)
5′-GACTAAGCTTCAATTGGATCCATGGACTAAATTAAAGTAATAAAGC
GTTCT

The approx. 1.1 kb amplified fragment was digested with MfeI+NcoI and ligated to MfeI+NcoI-digested pSJ12964. The ligation mixture was treated (amplified) using a TempliPhi polymerase kit according to the manufacturers instruction and this amplified ligation mixture was used for transformation of B. subtilis JA1343 competent cells. Spectinomycin resistant, chloramphenicol sensitive colonies were isolated and analysed by restriction digestion, and two correct clones kept, as SJ13016 (JA1343/pSJ13016) and SJ13017 (JA1343/pSJ13017).

Strains suitable for transfer of pSJ13016 and pSJ13017 by conjugation were constructed by introduction of the plasmids into B. subtilis PP3724 competent cells, selecting erythromycin resistance (2 microgram/ml). From each transformation, 4 colonies were pooled and frozen as follows:
SJ13042=PP3724/pSJ13016.
SJ13043=PP3724/pSJ13017.

### Example 5. Construction of Vectors pSJ13018 and pSJ13019 Carrying FRT-F-Cat::Kan-FRT-F3 Between catL Segments

In one approach to have a kan gene (conferring kanamycin resistance) flanked by FRT-F and FRT-F3 sites, the kan gene was inserted into (and thus inactivating) the FRT-flanked cat gene in pSJ12964, using the unique MfeI and NcoI sites within the cat gene. The kan gene was PCR amplified from plasmid pSJ3316 (U.S. Pat. No. 5,882,888) using primers #6450+#6451, which incorporated an upstream MfeI and a downstream NcoI site in the amplified fragment.

Primer B450:
(SEQ ID NO: 10)
5′-GACTGAATTCCATGGCAATTGGGCCAGTTTGTTGAAGATTAGA
Primer B451:
(SEQ ID NO: 11)
5′-GACTAAGCTTCAATTGGATCCATGGCCAACATGATTAACAATTATTA
GAG

The approx. 1 kb amplified fragment was digested with MfeI+NcoI and ligated to MfeI+NcoI-digested pSJ12964. The ligation mixture was treated (amplified) using a TempliPhi polymerase kit according to the manufacturers instruction and this amplified ligation mixture was used for transformation of B. subtilis JA1343 competent cells. Kanamycin resistant, chloramphenicol sensitive colonies were isolated and analysed by restriction digestion, and two correct clones kept as SJ13018 (JA1343/pSJ13018) and SJ13019 (JA1343/pSJ13019).

Strains suitable for transfer of pSJ13018 and pSJ13019 by conjugation were constructed by introduction of the plasmids into B. subtilis PP3724 competent cells, selecting erythromycin resistance (2 microgram/ml). From each transformation, 4 colonies were pooled and frozen as follows:
SJ13044=PP3724/pSJ13018.
SJ13045=PP3724/pSJ13019.

### Example 6. Construction of Vectors pSJ13046, and -47 Carrying FRT-F-PamyL_4199-dsRED-FRT-F Between catL Segments

Plasmid pPP4758 was used as a PCR template for amplification of the dsRED gene. To have the dsRED gene flanked both upstream and downstream by FRT-F sites, oligonucleotides incorporating these sites were designed for amplification of the dsRED gene. Oligonucleotide #B435 is a forward primer for the dsRED gene segment, incorporating a FRT-F site and restriction sites EcoRI, BamHI, and SalI for cloning, whereas oligonucleotide #6436 is a reverse primer for the dsRED gene segment, incorporating a FRT-F site and restriction sites HindIII, BamHI, XbaI, SalI, and EspI for cloning.

Primer B435:
(SEQ ID NO: 12)
5′-GACTGAATTCGGATCCGTCGACGAAGTTCCTATTCCGAAGTTCCTAT
TCTCTAGAAAGTATAGGAACTTCATAAATGAGTAGAAAGCGCCATATC
Primer B436:
(SEQ ID NO: 13)
5′-GACTAAGCTTGGATCCTCTAGAGTCGACGCTTAGCGAAGTTCCTATA
CTTTCTAGAGAATAGGAACTTCGGAATAGGAACTTCCTTTAGTGTCAATT
GGAAGTCTG

The dsRED gene from plasmid pPP4758 was PCR amplified using primers B435+B436, and the resulting appr 1 kb fragment digested with SalI. This SalI fragment was ligated to the 5.3 kb SalI digested pSJ8017, the ligation mixture introduced into B. subtilis JA1343 competent cells, and two resulting strains, forming red colonies indicating production of the dsRED protein, were kept as SJ13046 (JA1343/pSJ13046) and SJ13047 (JA1343/pSJ13047). The PCR amplified insert on these plasmids was confirmed correct by DNA sequencing.

The DNA sequence of the FRT-F-PamyL_4199-dsRED-FRT-F containing SalI insert in pSJ13046 is shown in SEQ ID NO:14.

Strains suitable for transfer of pSJ13046 and pSJ13047 by conjugation were constructed by introduction of the plasmids into B. subtilis PP3724 competent cells, selecting erythromycin resistance (2 microgram/ml). From each transformation, 4 colonies were pooled and frozen as follows:
SJ13058=PP3724/pSJ13046.
SJ13059=PP3724/pSJ13047.

### Example 7. Construction of Vector pSJ13048 Carrying FRT-F-PamyL_4199-dsRED-FRT-F3 Between catL Segments

Plasmid pPP4758 was used as a PCR template for amplification of the dsRED gene. To have the dsRED gene flanked upstream and downstream by different FRT sites, oligonucleotides incorporating these sites were designed for amplification of the dsRED gene. Oligonucleotide #B435 is a forward primer for the dsRED gene segment, incorporating a FRT-F site and restriction sites EcoRI, BamHI, and SalI for cloning, whereas oligonucleotide #B437 is a reverse primer for the dsRED gene segment, incorporating a FRT-F3 site and restriction sites HindIII, BamHI, XbaI, SalI, and EspI for cloning.

Primer B435: See example 6.
Primer B437:
(SEQ ID NO: 15)
5′-GACTAAGCTTGGATCCTCTAGAGTCGACGCTTAGCGAAGTTCCTATA
CTATTTGAAGAATAGGAACTTCGGAATAGGAACTTCCTTTAGTGTCAATT
GGAAGTCTG

The dsRED gene from plasmid pPP4758 was PCR amplified using primers B435+B437, and the resulting appr 1 kb fragment digested with SalI. This SalI fragment was ligated to the 5.3 kb SalI digested pSJ8017, the ligation mixture introduced into B. subtilis JA1343 competent cells, and a resulting strain, forming red colonies, was kept as SJ13048 (JA1343/pSJ13048). DNA sequencing confirmed the correctness of the FRT-F and FRT-F3 segments on this plasmid.

A strain suitable for transfer of pSJ13048 by conjugation was constructed by introduction of the plasmid into B. subtilis PP3724 competent cells, selecting erythromycin resistance (2 microgram/ml). 4 colonies were pooled and frozen as SJ13060=PP3724/pSJ13048.

### Example 8. Integration of FRT-F-Cat::Marker-FRT-F3 Segments into the B. licheniformis catL Locus

Donor strains SJ13037 to SJ13041 (having constructs with FRT-F-cat::dsRED-FRT-F3 between catL segments), SJ13042-43 (having constructs with FRT-F cat::spc-FRT-F3 between catL segments), and SJ13044-45 (having constructs with FRT-F-cat::kan-FRT-F3 between catL segments) were used in conjugations (essentially as previously described) to each of strains SJ12850 and SJ12851, selecting for erythromycin resistance (2 microgram/ml).

Transconjugants were spread onto LBPGS plates with erythromycin (2 microgram/ml), and these incubated at 50° C. to select for strains with chromosomal integration of the plasmids. Subsequently, such colonies were propagated for a number of transfers on LBPSG plates at 34° C. and replicated to look for erythromycin sensitive isolates that had lost the integrated plasmid but retained either the red color (indicating presence of dsRED), or spectinomycin or kanamycin resistance, as appropriate. Eventually, a number of strains were isolated:
SJ13049: SJ12851/SJ13037: SJ12851 3′ catL, FRT-F, cat::dsRed, FRT-F3, 5′ catL.
SJ13055: SJ12850/SJ13045: SJ12850 3′ catL, FRT-F, cat::kan, FRT-F3, 5′ catL
SJ13056: SJ12850/SJ13045: SJ12850 3′ catL, FRT-F, cat::kan, FRT-F3, 5′ catL
SJ13057: SJ12851/SJ13045: SJ12851 3′ catL, FRT-F, cat::kan, FRT-F3, 5′ catL
SJ13096: SJ12850/SJ13037: SJ12851 3′ catL, FRT-F, cat::dsRed, FRT-F3, 5′ catL.
SJ13097: SJ12850/SJ13041: SJ12851 3′ catL, FRT-F, cat::dsRed, FRT-F3, 5′ catL.
SJ13098: SJ12850/SJ13042: SJ12850 3′ catL, FRT-F, cat::spc, FRT-F3, 5′ catL
SJ13099: SJ12850/SJ13043: SJ12850 3′ catL, FRT-F, cat::spc, FRT-F3, 5′ catL
SJ13100: SJ12851/SJ13042: SJ12851 3′ catL, FRT-F, cat::spc, FRT-F3, 5′ catL
SJ13101: SJ12851/SJ13043: SJ12851 3′ catL, FRT-F, cat::spc, FRT-F3, 5′ catL

### Example 9. Integration of a FRT-F-PamyL_4199-dsRED-FRT-F Segment into the B. licheniformis catL Locus

The vectors pSJ13046 and -47, carrying a FRT-F-PamyL_4199-dsRED-FRT-F segment between catL up- and downstream fragments, were introduced into B. licheniformis strains SJ13055, -56, and -57 (having a chromosomally integrated FRT-F, cat::kan, FRT-F3 segment between catL up- and downstream fragments), and into B. licheniformis strains SJ12850 and -51 (having chromosomally integrated catL up- and downstream fragments), by conjugation from B. subtilis donor strains SJ13058 and -59 essentially as previously described, and followed by chromosomal integration/excision of the introduced plasmid via the catL up- and downstream segments, common to the plasmid and the host strain.

Integrant strains were obtained after plating and colony formation on LBPGS agar plates with erythromycin (2 microgram/millliliter) at 50° C., then propagation of such colonies on LBPGS agar plates without antibiotics at 34° C. to allow plasmid replication and excision from the chromosome, if necessary for several transfers, plating for single colonies, and isolation of resulting erythromycin sensitive and red pigmented strains. For strains derived from the SJ13055-57 hosts, candidate colonies were also tested for kanamycin sensitivity.

Resulting strains, containing in their chromosomes a FRT-F-PamyL_4199-dsRED-FRT-F segment between catL up- and downstream fragments, were kept as:
SJ13103: from SJ13055 by integration/excision of pSJ13047 introduced by conjugation from SJ13059.
SJ13104: from SJ13055 by integration/excision of pSJ13047 introduced by conjugation from SJ13059.
SJ13105: from SJ13055 by integration/excision of pSJ13047 introduced by conjugation from SJ13059.
SJ13106: from SJ13055 by integration/excision of pSJ13047 introduced by conjugation from SJ13059.
SJ13114: from SJ12850 by integration/excision of pSJ13046 introduced by conjugation from SJ13058.
SJ13115: from SJ12850 by integration/excision of pSJ13046 introduced by conjugation from SJ13058.
SJ13116: from SJ12850 by integration/excision of pSJ13046 introduced by conjugation from SJ13058.

### Example 10. Integration of a FRT-F-PamyL_4199-dsRED-FRT-F3 Segment into the B. licheniformis catL Locus

The vector pSJ13048, carrying a FRT-F-PamyL_4199-dsRED-FRT-F3 segment between catL up- and downstream fragments, were introduced into B. licheniformis strains SJ13055, -56, and -57 (having a chromosomally integrated FRT-F, cat::kan, FRT-F3 segment between catL up- and downstream fragments), and into B. licheniformis strains SJ12850 and -51 (having chromosomally integrated catL up- and downstream fragments), by conjugation from B. subtilis donor strain SJ13060 essentially as previously described, and followed by chromosomal integration/excision of the introduced plasmid via the catL up- and downstream segments, common to the plasmid and the host strain.

Integrant strains were obtained after plating and colony formation on LBPGS agar plates with erythromycin (2 microgram/millliliter) at 50° C., then propagation of such colonies on LBPGS agar plates without antibiotics at 34° C., to allow plasmid replication and excision from the chromosome, if necessary for several transfers, plating for single colonies, and isolation of resulting, erythromycin sensitive and red pigmented strains. For strains derived from the SJ13055-57 hosts, candidate colonies were also tested for kanamycin sensitivity.

Resulting strains, containing in their chromosomes a FRT-F-PamyL_4199-dsRED-FRT-F3 segment between catL up- and downstream fragments, were kept as
SJ13107: from SJ13055 by integration/excision of pSJ13048 introduced by conjugation from SJ13060.
SJ13108: from SJ13057 by integration/excision of pSJ13048 introduced by conjugation from SJ13060.
SJ13109: from SJ13057 by integration/excision of pSJ13048 introduced by conjugation from SJ13060.
SJ13117: from SJ12851 by integration/excision of pSJ13048 introduced by conjugation from SJ13060.
SJ13118: from SJ12851 by integration/excision of pSJ13048 introduced by conjugation from SJ13060.

### Example 11. Integration of a FRT-F-Gfp-FRT-F3 Segment Downstream of a Strong Promoter at the B. licheniformis amyL Locus

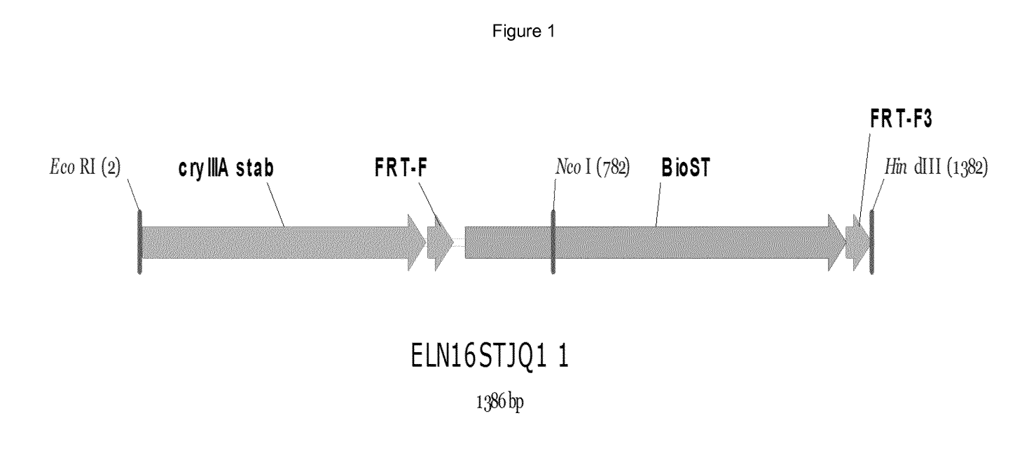

A DNA segment containing the mRNA stabilizing element from the Bacillus thuringiensis cry3A gene, followed by a FRT-F site, a green-fluorescent protein (gfp) CDS with a ribosome binding site, and a FRT-F3 site was designed as shown in FIG. 1; the sequence is provided in SEQ ID NO:16. The segment was ordered from a commercial supplier (GeneArt, DK) and inserted in their standard plasmid vector pMK-RQ, conferring kanamycin resistance.

The synthetic construct was received as pMK-RQ ELN16STJQ1_1, and introduced by electroporation into E. coli TG1 cells, selecting for growth at 40 microgram/ml kanamycin. Two strains were kept, as SJ13184 and SJ13185.

To transfer the construct into a vector enabling chromosomal integration in B. licheniformis, plasmid from pSJ13184 was digested with EcoRI+HindIII, and the 1.4 kb fragment gel purified. The fragment was ligated to the similarly purified 4.6 kb EcoRI-HindIII vector fragment from pSJ5487 (WO2005123915), the ligation mixture treated with TempliPhi (isothermal rolling circle amplification kit from GE Healthcare), and added to B. subtilis JA1343 competent cells, selecting for erythromycin resistance (2 microgram/ml) at 34° C. Among 8 colonies analysed, 5 were correct as based on restriction digests, and these 5 also exhibited green fluorescence. Two were kept, as SJ13193 (JA1343/pSJ13193) and SJ13194 (JA1343/pSJ13194).

Each of these plasmid preparations were introduced into the conjugative donor strain B. subtilis PP3724, and lots of transformants were obtained which were pooled and kept as SJ13195 (PP3724/pSJ13193) and SJ13196 (PP3724/pSJ13194).

Strain SJ13196 was used as donor in a conjugation to SJ12851, transconjugants were obtained and reisolated to single colonies on LBPGS 2 Erm at 50° C. There was a mix of green and colourless colonies, and three colourless colonies were propagated in TY medium for 3 days at 34° C. The cultures were then plated to single colonies on LBPGS plates, and from one culture predominantly amylase negative colonies were found. These were further reisolated and erythromycin sensitive, amylase negative, and strongly green fluorescing colonies were obtained. DNA from the amyL locus was PCR amplified, using primers #411451+#411452. A PCR fragment of the correct size confirming replacement of the amyL gene with the FRT-F-gfp-FRT-F3 construct was obtained, and sequencing using primers #411452, B539, B533, B471 and B335 confirmed insertion of the desired construct. 2 correct strains were kept, as SJ13235 and SJ13236.

### Primer Sequences:

#411451
(SEQ ID NO: 17)
5′-GTCCGAATCCCGCTACAACG
#411452
(SEQ ID NO: 18)
5′-CAATGACGTGACGTGTTGCC
#B335
(SEQ ID NO: 19)
5′-TTCTATTGGAATGATTAAGATTCCA
#B471
(SEQ ID NO: 20)
5′-GCACCGTCTAATGGATTTATGAA
#B533
(SEQ ID NO: 21)
5′-CTTCATTGCGGAATGAACAAGC
#B539
(SEQ ID NO: 22)
5′-TTGCCCGAATACAACGACAGGC

### Example 12. Construction of FLP Expression Vector pSJ13052

The amino acid sequence of a flippase (FLP) having one substitution at pos. 5 where a D has replaced a G present in yeast FLP (UNIPROT: P03870) was used as basis for the design of a synthetic coding sequence optimized for expression in B. licheniformis.

Two different synthetic sequences encoding the same amino acid sequence were designed, equipped with desired flanking sequences (a.o. the amyL RBS and the amyL transcriptional terminator) and ordered from a commercial supplier (GeneArt, Denmark).

One synthetic sequence, named P33JVS_r4_fl, was received as a DNA string, whereas another sequence, named D438GY_fl, was received inserted in a standard Geneart vector conferring kanamycin resistance, which was introduced into E. coli SJ2 by electroporation and two transformants, selected on LBPGS plates with 40 microgram/ml kanamycin, were kept as SJ12975 and SJ12976. The nucleotide sequence of P33JVS_r4_fl is shown in SEQ ID NO:23 and the sequence of D438GY_fl is shown in SEQ ID NO:24.

The synthetic FLP-encoding construct D438GY_fl was transferred to a mobilizable, temperature-sensitive Bacillus vector as follows: pSJ12975 was used as template in a PCR amplification using primers #B440+#B441. The amplified fragment digested with MscI+MluI, and the gel purified, digested fragment was ligated to the similarly digested, gel purified vector fragment of 5.3 kb from pSJ9806. The ligation mixture was transformed into B. subtilis JA1343 competent cells, selecting for erythromycin resistance (2 microgram/ml) at 30° C. 16 transformants were obtained, 8 were analyzed by restriction digests, 7 deemed correct, these analyzed by DNA sequencing, all confirmed correct, and three kept as:
SJ13052 (JA1343/pSJ13052)
SJ13053 (JA1343/pSJ13053)
SJ13054 (JA1343/pSJ13054)

pSJ13052 was introduced into the conjugative donor host strain PP3724, resulting in SJ13063, which can be used to transfer pSJ13052 into B. licheniformis.

### Primer Sequences:

#B440:
(SEQ ID NO: 25)
5′-GACTGGATCCGAATTCCAATTG
#B441
(SEQ ID NO: 26)
5′-AGTCGGATCCGAATTCAAGCTTG

### Example 13. Construction of a Vector with FLP and F-Cat-F3, pSJ13216

In a first step, the cry3A_stab-D438GY_flp coding sequence was excised from pSJ13052 as a 1.9 kb EcoRI-SalI fragment and purified after agarose gel electrophoresis. The fragment was ligated to the similarly purified 2.7 kb EcoRI-SalI fragment from pUC19. The ligation mixture was introduced into E. coli TG1 by electroporation, selecting for ampicillin resistance and a correct transformant, as judged by restriction digests, was kept as SJ13131 (TG1/pSJ13131).

In a second step, a FRT-F-cat-FRT-F3 segment was excised as a 1.0 kb SalI fragment from pSJ12964 and purified after agarose gel electrophoresis. The fragment was ligated to the similarly purified 4.6 kb SalI linearized pSJ13131 fragment and introduced into E. coli TG1 by electroporation, selecting for ampicillin resistance. Plates with transformants were subsequently replicated to plates with 10 microgram/ml chloramphenicol, and a chloramphenicol resistant strain was isolated.

Restriction digests indicated that it contained a plasmid where the cat gene of the FRT-F-cat-FRT-F3 segment had been inserted in the same orientation as the flp gene. The strain was kept as SJ13140 (TG1/pSJ13140).

In a third step, the assembly of cry3A_stab-D438GY_flp-FRT-F-cat-FRT-F3 was transferred from the E. coli plasmid pSJ13140 onto a temperature-sensitive, mobilizable Bacillus vector, by excision and purification by gel electrophoresis of the 3.0 kb EcoRI-HindIII fragment from pSJ13140, and ligation of this to the similarly purified 4.3 kb EcoRI-HindIII fragment from pSJ8017.

The ligation mixture was treated with TempliPhi polymerase and the resulting amplified ligation mixture was introduced into B. subtilis JA1343 competent cells, selecting for erythromycin resistance (2 microgram/ml) at 30° C. Several transformants having a correct restriction pattern were obtained, and one having the correct DNA sequence of the EcoRI-HindIII insert segment was kept, as SJ13216 (JA1343/pSJ13216).

For later use in B. licheniformis, pSJ13216 was introduced into the conjugative B. subtilis PP3724 donor strain by competent cell transformation, selecting for erythromycin resistance (2 microgram/ml) on LBPSG plates containing D-alanine (100 microgram/ml), resulting in strain SJ13217.

### Example 14. Demonstration of FLP-Mediated Marker Deletion in B. licheniformis (A)

B. subtilis strain SJ13063 is a conjugative donor strain containing pSJ13052, a plasmid carrying a construct with cry3A_stab-D438GY flp-dws_amyL, for FLP protein expression. This strain was used as donor in conjugations to B. licheniformis strains SJ13103 (chromosomal FRT-F-PamyL_4199-dsRED-FRT-F), SJ13107 and -08 (FRT-F-PamyL_4199-dsRED-FRT-F3).

Transconjugants were selected on LBPGS plates with erythromycin (2 microgram/millilitre) at 34° C. Transconjugant colonies in recipient SJ13103 were a mixture of red and colorless colonies, whereas transconjugants in SJ13107 and -08 were only red colonies. 15 colonies were picked at random from each of these initial transconjugant plates. These were all colorless from the SJ13103 recipient, and all red from the SJ13107 and -08 recipients.

One such colorless colony was characterized by PCR amplification which confirmed loss of the dsRED gene, and it was kept as SJ13123. The primer set used for PCR amplification was #B435+#B436 (both provided above), which should give a 1 kb fragment if the dsRED gene is present.

This illustrates the FLP-mediated deletion between two co-oriented FRT-F sites, and that no deletion was observed between similarly oriented FRT-F and FRT-F3 sites in B. licheniformis.

In a subsequent eksperiment, B. licheniformis strains SJ13103 to SJ13109, and SJ13114 to SJ13118 were used as recipients in conjugations from SJ13063, selecting for erythromycin resistance (2 microgram/ml). Transconjugants (15 picked at random from each transconjugant selection plate) in hosts SJ13103 to SJ13106 (FRT-F-PamyL_4199-dsRED-FRT-F) were all colourless, whereas transconjugants in SJ13107-09 (FRT-F-PamyL_4199-dsRED-FRT-F3) were all red.

Similarly, transconjugants from hosts SJ13114-16 (FRT-F-PamyL_4199-dsRED-FRT-F) were all colourless, whereas transconjugant colonies picked from hosts SJ13117 and -18 (FRT-F-PamyL_4199-dsRED-FRT-F3) were red.

The colourless strains were all confirmed, by PCR amplification as above, to have lost the dsRED gene

This confirms the above finding: successful FLP-mediated deletion between two co-oriented FRT-F sites, and no deletion observed between similarly oriented FRT-F and FRT-F3 sites in B. licheniformis.

Some exemplary transconjugants were kept as
SJ13124: SJ13104+SJ13063, colourless.
SJ13125: SJ13114+SJ13063, colourless.
SJ13126: SJ13115+SJ13063, colourless.
SJ13127: SJ13116+SJ13063, colourless.

### Example 15. Demonstration of FLP-Mediated Marker Replacement in B. licheniformis (B)

To achieve FLP expression in a host strain containing a chromosomal FRT-F-PamyL_4199-dsRED-FRT-F3 segment, pSJ13052 was introduced into each of SJ13107 and SJ13108 by conjugation from SJ13063, selecting for erythromycin resistance (2 microgram/ml). Two transconjugants in each host was kept:
SJ13110 and SJ13111 (SJ13107/pSJ13052)
SJ13112 and SJ13113 (SJ13108/pSJ13052).

Each of strains SJ13110 to SJ13113 were used as recipients in conjugations with SJ13042 and SJ13043, selecting for spectinomycin resistance (180 microgram/ml), and with SJ13044 and SJ13045, selecting for kanamycin resistance (40 microgram/ml). After several re-isolations on plates with antibiotics, some strains were isolated which exhibited the relevant antibiotic resistance, but were colourless, indicating loss of the dsRED gene as confirmed by PCR amplifications. Some exemplary strains were kept as:
SJ13120: SpcR, colourless isolate from SJ13113 following introduction of pSJ13016 by conjugation from SJ13042.
SJ13121: SpcR, colourless isolate from SJ13113 following introduction of pSJ13016 by conjugation from SJ13042.
SJ13122: SpcR, colourless isolate from SJ13110 following introduction of pSJ13017 by conjugation from SJ13043.
SJ13128: KanR, colourless isolate from SJ13110 following introduction of pSJ13018 by conjugation from SJ13044.

### Example 16. Demonstration of FLP-Mediated Marker Replacement in B. licheniformis (C)

FLP-mediated marker replacement was also demonstrated in host strains SJ13235 and SJ13236 described above in example 11, which very strongly express a Green Fluorescent Protein as a consequence of integration of a FRT-F-gfp-FRT-F3 segment downstream of the strong triple promoter system present at the amyL locus. The high expression level makes loss of the gfp gene easily detectable. Gene replacement resulted from introduction of a vector, pSJ13216, which carries both the flp gene and a FRT-F-cat-FRT-F3 segment.

Strains SJ13235 and SJ13236 were used as recipients in conjugations with donor strain SJ13217, the conjugation plates were replica plated to LBPGS+2 microgram/ml erythromycin, and the replica plates incubated at 34° C. for 2 days. 4 colonies from each strain combination were then inoculated into TY+5 microgram/ml erythromycin and shaken for 3 days at 33° C.

Aliquots from each tube were subsequently reisolated for single colonies on LBPSG plates with either 6, 10, 20, and 40 microgram/ml chloramphenicol, incubated at 37° C., or on LBPGS+2 microgram/ml erythromycin, incubated at 34° C.

Two days later, colourless colonies were found on all plates, many on the plates containing erythromycin, and even more, relative to green colonies, on plates with either 6, 10 or 20 microgram/ml chloramphenicol. There was only growth at the application area on the 40 microgram/ml chloramphenicol plates, indicating this antibiotic concentration is too high for proper selection.

Colonies from the 6 cam and 10 cam plates were replicated to cam and erm, and 15 colourfree, camR, and ermS colonies were reisolated further at 34° C. and confirmed, by PCR using a cat-internal primer (B321) together with an amyL locus downstream primer (#411452), to have had the cat gene inserted, in stead of the gfp gene, between the FRT-F and FRT-F3 sites at the chromosomal amyL locus. Some exemplary colourfree, camR strains were kept as SJ13242, SJ13243, SJ13244, and SJ13245.

Similarly, colourfree colonies were reisolated from the LBPGS+2 microgram/ml erythromycin plates onto plates without erythromycin. These plates were subsequently replica-plated to LBPSG+/−erm, and colourfree, erythromycin sensitive colonies were further reisolated, confirmed by PCR as above and, using also an amyl upstream (#411451)+a cat internal primer (#B317), were also confirmed to have the cat gene inserted in stead of the gfp gene between the FRT-F and FRT-F3 sites at the chromosomal amyL locus. They were also confirmed to exhibit chloramphenicol resistance (these colonies had not previously been selected on chloramphenicol). Two exemplary strains were kept as SJ13246 and SJ13247.

Thus, this example demonstrates a very efficient FLP-mediated marker replacement in lichenformis.

Primer B317:
(SEQ ID NO: 27)
5′-GTTTTATGTTTCGGTATAAAACAC
Primer B321:
(SEQ ID NO: 28)
5′-TATTCCATGGACTTCATTTACTG

### Example 17. Demonstration of FLP-Mediated Marker Replacement in B. licheniformis (D)

To achieve FLP expression in a host strain containing a chromosomal triple promoter-FRT-F-gfp-FRT-F3 segment, pSJ13052 was introduced into each of SJ13235 and SJ13236 by conjugation from SJ13063, selecting for erythromycin resistance (2 microgram/ml). One transconjugant in each host was kept as SJ13248 (SJ13235/pSJ13052) and SJ13249 (SJ13236/pSJ13052).

These transconjugant strains were used as recipient strains in conjugations with SJ13044, where transconjugants were selected using 40 microgram/ml kanamycin. Some colourless transconjugants were obtained, these were further reisolated and ErmS strains were obtained, and some of these were confirmed by PCR amplification/sequencing to have the kanR gene inserted in the chromosome replacing the GFP gene. Two exemplary strains, derived from SJ13248, were kept as: SJ13264 and SJ13265.

### Example 18. Construction of a Host Strain SJ13615 for Insertion of 3 Copies of Genes of Interest Using Flp Mediated Marker Replacement in B. licheniformis

A Bacillus licheniformis host strain was developed for achieving insertion of 3 copies of genes of interest. The strain was developed in the same strain line as strain SJ8071, and has 3 cassettes expressing Red Fluorescent Protein from FRT-F and FRT-F3 flanked constructs integrated after a strong tandem triple promoter at the bglC, the xylA, and the lacA2 loci, constructs designed to achieve a similar result to the one described in example 11 for GFP insertion at the amyL locus (i.e. strong, triple promoter driven expression of the RFP marker protein).

An exemplary DNA sequence of a FRT-F and FRT-F3 flanked RFP expression construct integrated at the bglC locus is provided in SEQ ID NO:29.

The strain has, in addition to the modified loci described above, deletions in the amyL, the aprL, the mprL, the catL, the cypX, the ggt, the gntP, the sacB, and the spollAC loci, and has an inactivating mutation at the forD locus. None of these additional modifications have any relevance for the demonstration of flp mediated marker replacement.

### Example 19. Insertion of 3 Copies of Genes of Interest Using Flp Mediated Marker Replacement in B. licheniformis

A flp expression vector carrying also FRT-F-cat-FRT-F3 was described in example 63, pSJ13216. This vector was improved to achieve a better functionality (higher frequency of recombination) by replacement of the cry3A_stab segment present upstream of the flp gene with a small promoter sequence derived from Bacillus amyloliquefaciens, PamyQ(sc). This was done by a “Prolonged Overlap Extension” PCR strategy fusing a PCR fragment made using primers B651+B653 on SJ13235 genomic DNA as template, with a PCR fragment made using primers B622+B664 using pSJ13216 as template.

The resulting improved vector, introduced into B. subtilis competent cells, was kept as SJ13461.

### Primer Sequences:

#B651:
(SEQ ID NO: 30)
5'-GACTAGATCTGAATTCTGCTGTCCAGACTGTCCGCTG
#B653:
(SEQ ID NO: 31)
5'-GACTAGATCTGGCCACATTTTCTTATACAAATTATATTATACATATC
#B622:
(SEQ ID NO: 32)
5'-AGTATCATATTGACGGCTTC
#B664:
(SEQ ID NO: 33)
5'-AATTTGTATAAGAAAATGTGGCCAGATCTAGTCCCACATTGAAAGGG
GAGGAGA

To enable an easy and versatile vector construction strategy, resulting in Bacillus vectors carrying flp and FRT-F-GOI-FRT-F3, a synthetic DNA segment was designed, by which BspQ1 sites were built into a small vector segment placed between FRT-F and FRT-F3, so that the recognition sites themselves would be cut away by digestion of the vector, leaving a vector fragment looking like: . . . Bacillus vector-pAmyQ(sc)-flp-FRT-F-BspQ1 digested end - - BspQ1 digested end-FRT-F3-Bacillus vector . . . .

Suitable primers may then be designed, that allows PCR amplification and BspQ1 digestion of a desired RBS-GOI fragment, which fragment after BspQ1 digestion can be ligated to the above vector fragment.

Such a vector is pSJ13654, having the DNA sequence given in SEQ ID NO:34.

Vector pSJ13654 was used for cloning of an amylase gene, amyL, by PCR amplification from Bacillus licheniformis, using primers B692 and B693 suitable for the BspQ1 cloning strategy. The ligation mixture was introduced into a Bacillus subtilis conjugative donor strain, saved as SJ13661, and used for conjugation into SJ13615. Transconjugants were selected on plates with erythromycin (2 microgram/ml), and suspensions subsequently diluted and plated to single colonies, without erythromycin.

The host has 3 RFP expression cassettes, to be replaced by the amyL gene. In first round, strains were isolated that were reduced in their red color intensity, and at the same time expressed amylase. One such was kept as SJ13666, carrying 1 or 2 copies of the amylase insert. This strain was used as host for a new round of conjugation with donor strain SJ13661, and colorless, amylase positive strains isolated, and kept as SJ13737 and SJ13738.

PCR across the integration loci (bglC, xylA, lacA2), and subsequent DNA sequencing, confirms the introduction of an amylase gene at each of the 3 loci.

### Primer Sequences:

#B692:
(SEQ ID NO: 35)
5′-GACTGCTCTTCCTTCATTGAAAGGGGAGGAGAATC
#B693:
(SEQ ID NO: 36)
5′-GACTGCTCTTCTCCTCTATCTTTGAACATAAATTG

1: A method for the site-specific integration of at least one polynucleotide of interest into the chromosome of a Bacillus licheniformis host cell, said method comprising the steps of:
(a) providing a B. licheniformis host cell comprising in its chromosome at least one integration site, each integration site comprising a pair of recognition sequences of the site-specific Flippase recombinase, FLP, from Saccharomyces cerevisiae, or a homologoue or variant thereof;
(b) introducing into said cell a nucleic acid construct also comprising the pair of recognition sequences of the site-specific recombinase, said pair flanking the polynucleotide of interest;
(c) expressing the site-specific FLP recombinase or homologue thereof in the cell, whereby the at least one chromosomal recognition sequence pair is recombined with the corresponding recognition sequence pair of the nucleic acid construct by the FLP recombinase to produce a B. licheniformis host cell comprising at least one polynucleotide of interest site-specifically integrated into the chromosome of the cell.

2: The method of claim 1, wherein the polynucleotide of interest comprises an operon or an open reading frame encoding at least one polypeptide of interest.

3: The method of claim 1, wherein the polypeptide of interest comprises an enzyme, preferably a hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

4: The method of claim 1, wherein a selection or screening marker is located in between the pair of recognition sequences in the at least one integration site.

5: The method of claim 1, wherein the nucleic acid construct further comprises an incoming selection marker and a polynucleotide encoding the FLP recombinase, or a homologue thereof.

6: The method of claim 1, wherein a second nucleic acid construct is introduced into said cell in step (b) which is either non-replicating or temperature-sensitively replicating, and which comprises a polynucleotide encoding the FLP recombinase, or a homologue thereof, and a selection marker which enables positive or negative selection or is bi-directional, and which is maintained in said cell transiently by selective pressure or growth at the permissive temperature, respectively, so that the recombinase can be transiently expressed in step (c).

7: The method of claim 1, wherein the cell in step (a) comprises in its chromosome at least one copy of a polynucleotide encoding the recombinase operably linked with a tightly regulated promoter, which can be turned on and off by changing the growth conditions, so as to enable the transient expression of the site-specific recombinase in step (c).

8: The method of claim 1, wherein the pair of recognition sequences consists of two different recognition sequences, preferably the wildtype FRT sequence in combination with a derivative thereof, more preferably the wildtype FRT sequence in combination with a recognition sequence selected from the group consisting of FRT-F, FRT-F3, FRT-F10, FRT-F13, FRT-F14, FRT-F15, FRT-Fa and FRT-F3a.

9: The method of claim 1, wherein the B. licheniformis host cell of step (a) comprises in its chromosome two or more integration sites and a B. licheniformis host cell comprising two or more polynucleotides of interest site-specifically integrated into the chromosome of the cell is produced; preferably the B. licheniformis host cell of step (a) comprises in its chromosome three or more integration sites; more preferably four or more, five or more, or even six or more integration sites, and wherein a B. licheniformis host cell comprising three, four, five, six or more polynucleotides of interest site-specifically integrated into the chromosome of the cell is produced.

10: A prokaryotic host cell comprising in its genome at least two polynucleotides encoding a polypeptide of interest, wherein each polynucleotide is flanked by a pair of recognition sequences for a site-specific recombinase.

11: The prokaryotic host cell of claim 10, which is a Gram-positive host cell; preferably, the prokaryotic host cell is a Bacillus host cell; more preferably, the prokaryotic host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells; even more preferably, the prokaryotic host cell is a Bacillus licheniformis host cell.

12: The prokaryotic host cell of claim 10, wherein the polypeptide of interest comprises an enzyme, preferably a hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, xylanase, or beta-xylosidase.

13: The prokaryotic host cell of claim 10, wherein the site-specific recombinase is Flippase (FLP) from Saccharomyces cerevisiae, or a homologoue or variant thereof.

14: The prokaryotic host cell of claim 10, wherein the pair of recognition sequences consists of two different recognition sequences; preferably the wildtype FRT sequence in combination with a derivative thereof; more preferably the wildtype FRT sequence in combination with a recognition sequence selected from the group consisting of FRT-F, FRT-F3, FRT-F10, FRT-F13, FRT-F14, FRT-F15, FRT-Fa and FRT-F3a.
